(19) United States
(12) Patent Application Publication
(10) Pub. No.: US 2005/0226830 A1
(43) Pub. Date: Oct. 13, 2005

(54) Nano pearl cream

(76) Inventor: Harvard Fang, City of Industry, CA (US)

Correspondence Address: LAW OFFICES OF CLEMENT CHENG, 17220 NEWHOPE STREET #127, FOUNTAIN VALLEY, CA, 92708, US

(21) Appl. No.: 10/819,822

(22) Filed: Apr. 7, 2004

(51) Int. Cl. A61K 7/021; A61K 7/06; A61K 35/80; A61K 7/00; A61K 35/78

(52) U.S. Cl. 424/63; 424/74; 424/195.17; 424/746; 424/769

(57) ABSTRACT

A topical application comprises cultured pearl powder of particle size ranging between 0.4 μm and 1 μm ground by an ultrasonic gas jet mill, and purified water suspending the cultured pearl powder.

### DISCUSSION OF RELATED ART

Pearl powder used on the face has been discussed in legends of Chinese Empresses as long as 2000 years ago. The pearl offers minerals that when applied topically provide some ultraviolet protection, moisture and amino acids to skin keep the skin smooth and supple.

In the past 30 years, researchers have been actively searching for means to block and shield radiation exposure. The prior art contains several skin care or cosmetic compositions of different ingredients, which include, among other things, Mother of Pearl. Mother of Pearl is the material that comprises the inner lining of a mollusk shell. These compositions are used to treat damaged skin or to improve the appearance of skin.

U.S. Pat. No. 4,393,045 discloses a powder form of Mother of Pearl obtained by blending, crushing, pulverizing, pestle, and the like. U.S. Pat. No. 5,085,870 also discloses an ingredient as extracted from Mother of Pearl. U.S. Pat. No. 5,773,034 discloses specifically “pearl testa of the bivalved mollusc Pinctada maxima” and grinds said substance into powder form. U.S. Pat. No. 5,538,740 discusses using secretions of fasted snails to be mixed with different calcium carbonate ingredients in powder form. Unfortunately, current topical applications have short efficacy and questionable strength.

### OBJECTS AND ADVANTAGES OF THE INVENTION

a. Ultra-fine particle: The average particle size has been measured to be 0.4 μM by nano-meter technology.
b. Apparent efficacy: These ultra-fine particles have been proven to effectively shield or block 50 to 90% of radiation.
c. Easy to use: Our products can be easily and readily used in comparison to similar or comparable competitors cosmetic products.
d. A matured technology: The “super sonic air jet pulverization” process making the ultra-fine pearl powder has been refined such that the final product particle size can be fully controlled and duplicated.
e. Widely used ingredient: Pearl powder has been used in various different cosmetic products, particularly in cosmetic, therapeutic, health care and skin care products.
f. Innocuous and non-toxic: It is a completely non-toxic and innocuous to humans. On the contrary, it contains certain essential nutritional elements with cosmetic effects.

### SUMMARY OF THE INVENTION

The present invention provides a cosmetic composition formed by intimately admixing the nano-meter sized ultra-fine pearl particles with shielding and blocking ingredients. The nano-meter sized ultra-fine pearl particles are formed by super sonic air jet pulverization.

This cosmetic composition formula provides superior radiation blocking and shielding protection when applied topically to humans. The small particle size assists with adhesion and retention of pearl powder on skin.

### BRIEF DESCRIPTION OF THE DRAWING

FIG. 1 is an exemplary and non-limiting preferred embodiment of the invention, showing a flow block chart in accordance with the invention

### CALL OUT LIST OF THE STEPS

1. Ultra Fine Freshwater Pearl
2. Supersonic Air Jet Pulverization Process
3. Natural Pearl Powder
4. Lotion Ingredients
5. Mixing and Forming Process
6. Radiation Blocking and Shielding Products

### DETAILED DESCRIPTION OF THE INVENTION

Current commercially available supersonic grinding machines should be able to produce suitable sized pearl powder from cultured pearls. Ultra-fine 0.4 micron pearl powder nano particles can be created in super sonic air jet pulverization processes. Collisions between particles of pearl in supersonic grinding and classification machines begin with highly pressurized gas exit nozzles creating a supersonic grinding stream. Solid particles are injected into a supersonic stream. The stream being turbulent and entrained with particles provides high velocity collisions between solid particles in the stream. When a user introduces solid particles, supersonic flow forces solid particles into high velocity collisions forming micron particles and smaller than micron particles. As the particles are reduced in size, they can be separated from the main gas stream. A classifier traps properly sized product in viscous drag of exiting flow. The powder is removed to collection equipment. Gas used can include air or steam.

These present compositions are formulated and blended with ultra-fine pearl powder with an average nano-meter particle size and use such nano-meter sized ultra-fine pearl particles as major ingredients in different embodiments, such as facial creams, hand creams, cleansers, toners, masks, and serums etc.

This invention can be applied to human skin as a thin layer on the surface of the face, neck, and the palm and back of hands. This thin coating provides radiation protection. The smaller than micron sized particles can be mixed with a base such as glycerin. Glycerin evaporating rapidly provides uniform distribution of topically applied particles.

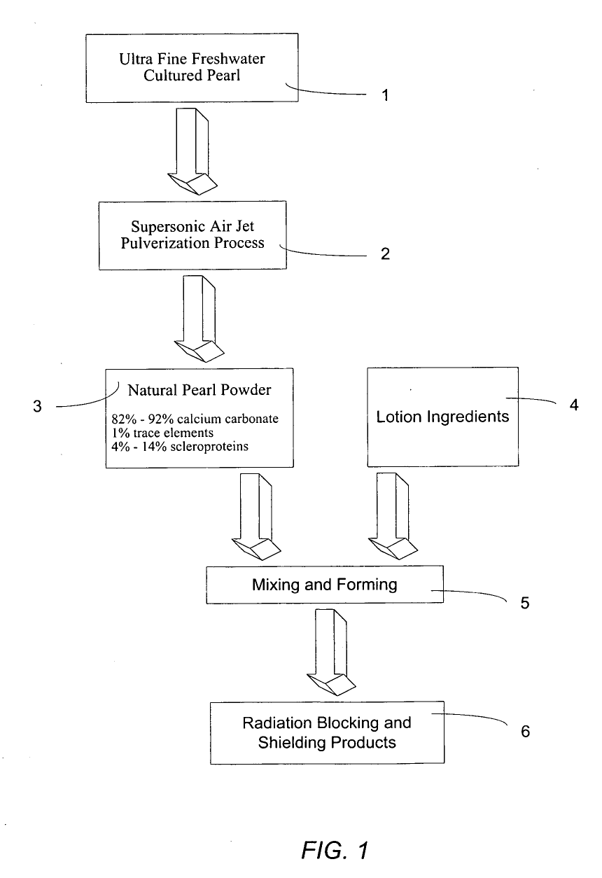

Referring to FIG. 1, the present invention provides cosmetic compositions formed by intimately admixing the nano-meter sized ultra-fine pearl particles with shielding and blocking ingredients; the nano-meter sized ultra-fine pearl particles are formed by “super sonic air jet pulverization”.

The natural pearl powder is processed from freshwater cultured pearl and its chemical composition is non-toxic and innocuous to humans. After pulverization, its ultra-fine size provides complete coverage when applied as skin care cream or lotion. It can be easily and readily absorbed through the skin.

All these ultra-fine pearl particles are in nano-meter size and formed by using a “Supersonic Air Jet Pulverization” process. The ultra-fine pearl particles formed by the “Supersonic Air Jet Pulverization” process are in a fraction of nano-meters. The major radiation-absorbing ingredient in this invention is this ultra-fine pearl particle that has an average size of 0.4 μM in diameter. One μM is equivalent to 1.0×10⁻⁴M. This particle is smaller than the skin epithelial cells in magnitude. On average, the epithelial cells are in the range of 5 to 8 uM, which would take 12 to 20 ultra-fine pearl particles to cover. Thus, each epithelial cell is covered by more than 10 pearl particles that are proven to shield and block radiation. Fine pearl particles are well distributed for covering skin. Also thin film application allows less use of material, and more uniform distribution.

The natural pearl powder comprises 82 to 92% calcium carbonate, approximately 1% trace elements and 4 to 14% scleroproteins. Scleroproteins are insoluble and have fibrous structure which is a major component of animal nails, and horns. This natural pearl powder is 100% soluble in water. It is non-toxic and innocuous to humans. When absorbed by human body, it can be absorbed and digested as nutrients with trace essential elements such as potassium, magnesium, copper, and zinc. The ultra-fine pearl powder can shield 50% to 90% ultraviolet radiation that would provide an SPF between 4 and 10.

The present invention provides a cosmetic composition formed by intimately admixing the nano-meter sized ultra-fine pearl particles with shielding and blocking ingredients. Such composition can be implied into different embodiments based upon the ingredients applied. Listed below are the formulations and various combinations of the ingredients for the following possible embodiments, among others, can be comprised by the present invention. Said ingredients often have different identities in practice. For example Glycerin can also be known as Glycyl alcohol. Also listed below is a list of most common synonyms, but not limited to, used in the market for reference.

1. Ingredients of the Embodiments

a. PearLife Fantastic O's Day Cream
Purified Water, Glycerin, C12-15 Alkyl Benzoate, Ethylhexyl Palmitate, Ethythexyl Methoxycinnamate, Glyceryl stearate, PEG-100 Stearate, Cetyl Alcohol, Stearic Acid Dimethicone, Cylomethicone, Dimethiconol, Carbomer, Disodium EDTA, Panthenol, Allantoin, Benzophenone 3, Tocopheryl Acetate, Triethanolamine, Natural Pearl Powder, Propylene Glycol, Methylparaben, Propylparaben, Diazolidinyl Urea, Fragrance

b. PearLife Fantastic O's Night Cream
Purified water, Butylenes Glycol, Panthenol, Ethylhexyl Palmitate, C12-15 Alkyl Benzoate, Glyceryl Stearate, Peg-100 Stearate, Cetyl Alcohol, Stearic Acid, Emusifying Wax, Natural Pearl Powder, Dimethicone, Tocopheryl Acetate, Retinyl Palmitate, Carbomer, Cyclomethicone, Dimethiconol, Aloe Barbadenbsis Gel, Algae Extract, Triethanolamine, Disodium EDTA, Mica, Propylene Glycol, Methyparaben, Propylparaben, Diazolidinyl Urea, Fragrance

c. PearLife Fantastic O's Cleanser Water, PEG-8, Glycerin, Sodium Laureth Sulfate, Potassium Cocoate, Sodium Lauryl Sulfate, Cetyl Alcohol, Glyceryl Stearate, Propylene Glycol Dicaprylate/Dicaprate, Allantoin, Hydroxyethylcellulose, Panthenol, Tocopheryl acetate, Natural Pearl Powder, Methylparaben, Propylparaben, Fragrance

d. PearLife Fantastic O's Toner
Water, Polysorbate 20, Natural Pearl Powder, Glycerin, Hydroxyethylcellulose, Panthenol, Aloe barbadensis Leaf Juice, Hamamelis Virginiana (Witch Hazel) Extract, Salvia Officinalis (Sage) Leaf Extract, Algae Extract, Citric Acid, Methylparaben, DMDM Hydantoin, Fragrance

e. PearLife Fantastic O's Eye Cream
Water, C12-15 Alkyl Benzoate, Ethylhexyl Palmitate, Glyceryl Stearate (and) PEG-100 Stearate Cetyl Alcohol, Dimethicone, Cyclomethicone, Stearic Acid, Glycerin, Emulsifying Wax, Natural Pearl Powder, Hydrozyethylcellulose, Triethanolamine, DMDM Hydantoin, Carbomer, Allantoin, Panthenol, Tocopheryl Acetate, Salvia Officinalis (Sage) Leaf Extract, Anthemis Nobilis (Chamomile) Flower Extract, Algae Extract, Retinyl Palmitate, Methylparaben, Propylparaben

f. PearLife Fantastic O's Collagen Serum
Water, Glycerin, Soluble Collagen, Natural Pearl Powder, Hydroxyethlcellulose, Polysorbate 20, DMDM Hydantoin, Elastin, Panthenol, Aloe Barbadensis Leaf Juice, Algae Extract, Citric Acid, Methyparaben, Fragrance

g. PearLife Fantastic O's Vitamin C Powder
Natural Pearl Powder, Purified Water, Propylene Glycol, Glycerin, Silica Silyate, Polymethylsilsesquioxane, Methylparaben, Imidazolidinyl Urea

h. PearLife Fantastic O's Arbutin Powder
Natural Pearl Powder, Water, Glycerin, Silica Silylate, Vunyl Dimethicone/Methicone, Silesquiozane Crosspolymer, Arbutin, Methylparaben, Imidazolidinyl Urea, Hamamelis Virginiana Extract, SD Alcohol 39-C, Butylene Glycol, Dipotassium Glycyrrhizate, Allantoin, Tocopherol Acetate
   2. Detail Synonyms of the Ingredients

a. PearLife Fantastic O's Day Cream

Purified Water

Glycerin, also known as 1,2,3-Propanetriol, Glycyl alcohol; Propanetriol or Trihydroxypropane

C12-15 Alkyl Benzoate

Ethylhexyl Palmitate

Ethythexyl Methoxycinnamate

Glyceryl Stearate, well known as Stearin, 1-mono-; (1)-2,3-Dihydroxypropyl stearate;

Glycerol stearate, pure; Octadecanoic acid, 2,3-dihydroxypropyl ester; Stearin, or 1-mono-(8CI)

PEG-100 Stearate

Cetyl Alcohol, well known as Alcohol C-16; Cetostearyl alcohol; 1-Hexadecanol; 1-Hexadecyl alcohol; or Palmityl alcohol

Stearic Acid, also known as 1-Heptadecanecarboxylic acid; Octadecanoic acid; or n-Octadecanoic acid

Dimethicone, also known as Dimethylpolysiloxane (1); Dimethyl polysiloxane; Dimethyl polysiloxane, bis(trimethylsilyl)-terminated; Polydimethylsiloxane (2); Dimethicone 350 [USAN]

Cyclomethicone (and) Dimethieonol

Carbomer, also known as 2-Propenoic acid, homopolymer; Carbomer 934 [USAN]; Carbomer 940 [USAN]; Polyacrylic acid; Acrylic polymer(s); Carbomer 941 [USAN]; Acrylic acid, polymers; Acrysol 1mw-20X; Polymer of 2-propenoic acid, cross-linked with allyl ethers of pentaerythritol

Disodium EDTA, also known as Disodium N,N′-1,2-ethanediylbis(N-(carboxymethyl)glycine); Disodium edetate; Disodium ethylenediaminetetraacetate; Edetate disodium; Glycine, N,N′-1,2-ethanediylbis(N-(carboxymethyl)-, disodium salt; Edetate sodium [USAN]; Acetic acid, (ethylenedinitrilo)tetra-, disodium salt; Ethylene diamine tetraacetic acid, disodium salt

Panthenol, also known as Butanamide, 2,4-dihydroxy-N-(3-hydroxypropyl)-3,3-dimethyl-, (R)-; Butyramide, 2,4-dihydroxy-N-(3-hydroxypropyl)-3,3-dimethyl-, D-(+)-; D-Panthenol; Dexpanthenol; Propanolamine, N-pantoyl-; d-Pantothenyl alcohol; Butanamide, 2,4-dihydroxy-N-(3-hydroxypropyl)-3,3-dimethyl-, (2R)-

Allantoin, also known as (2,5-Dioxo-4-imidazolidinyl)urea; 5-Ureidohydantoin; Glyoxyldiureide; Hydantoin, 5-ureido-; Urea, (2,5-dioxo-4-imidazolidinyl)-

Benzophenone 3, also known as (2-Hydroxy-4-methoxyphenyl)phenylmethanone; 2-Hydroxy-4-methoxybenzophenone; Methanone, (2-hydroxy-4-methoxyphenyl)phenyl-; Oxybenzone; Benzophenone, 2-hydroxy-4-methoxy-

Tocopheryl Acetate, also known as 2H-1-Benzopyran-6-ol, 3,4-dihydro-2,5,7,8-tetramethyl-2-(4,8,12-trimethyltridecyl)-, acetate; 3,4-Dihydro-2,5,7,8-tetramethyl-2-(4,8,12-trimethyltridecyl)-2H-b-enzopyran-6-ol, acetate; DL-alpha tocopheryl acetate; or Vitamin E acetate

Triethanolamine, also known as 2,2′,2″-Nitrilotriethanol; Ethanol, 2,2′,2″-nitrilotri-; TEA; Nitrilotriethanol; TEA (amino alcohol); Triethylamine, 2,2′,2″-trihydroxy-; Triethylolamine; Trihydroxytriethylamine; Tris(2-hydroxyethyl)amine; Trolamine; 2,2′,2″-Nitrilotris(ethanol); Ethanol, 2,2′,2″-nitrilotris-

Natural Pearl Powder

Propylene Glycol (and) Methylparaben (and) Propylparaben (and) Diazolidinyl

Urea

Fragrance

b. PearLife Fantastic O's Night Cream

Purifled water

Butylenes Glycol

Panthenol, also known as Butanamide, 2,4-dihydroxy-N-(3-hydroxypropyl)-3,3-dimethyl-, (R)-; Butyramide, 2,4-dihydroxy-N-(3-hydroxypropyl)-3,3-dimethyl-, D-(+)-; D-Panthenol; Dexpanthenol; Propanolamine, N-pantoyl-; d-Pantothenyl alcohol; Butanamide, 2,4-dihydroxy-N-(3-hydroxypropyl)-3,3-dimethyl-, (2R)-

Ethylhexyl Palmirate C12-15 Alkyl Benzoate

Glyceryl Stearate, also known as Stearin, 1-mono-; (1)-2,3-Dihydroxypropyl stearate; Glycerol stearate, pure; Octadecanoic acid, 2,3-dihydroxypropyl ester; Stearin, or 1-mono-(8CI)

Peg-100 Stearate

Cetyl Alcohol, also known as Alcohol C-16; Cetostearyl alcohol; 1-Hexadecanol; 1-Hexadecyl alcohol; or Palmityl alcohol

Stearic Acid, also known as 1-Heptadecanecarboxylic acid; Octadecanoic acid; or n-Octadecanoic acid

Emusifying Wax

Natural Pearl Powder

Dimethicone, also known as Dimethylpolysiloxane (1); Dimethyl polysiloxane; Dimethyl polysiloxane, bis(trimethylsilyl)-terminated; Polydimethylsiloxane (2); Dimethicone 350 [USAN]

Tocopheryl Acetate, also known as 2H-1-Benzopyran-6-ol, 3,4-dihydro-2,5,7,8-tetramethyl-2-(4,8,12-trimethyltridecyl)-, acetate; 3,4-Dihydro-2,5,7,8-tetramethyl-2-(4,8,12-trimethyltridecyl)-2H-b-enzopyran-6-ol, acetate; DL-alpha tocopheryl acetate; or Vitamin E acetate

Retinyl Palmitate, also known as Retinol, all-trans-, palmitate; Retinol palmitate; Retinol, hexadecanoate; Retinyl hexadecanoate; or Vitamin A palmitate

Carbomer, also known as Carbomer; 2-Propenoic acid, homopolymer; Carbomer 934 [USAN]; Carbomer 940 [USAN]; Polyacrylic acid; Acrylic polymer(s); Carbomer 941 [USAN]; Acrylic acid, polymers; Acrysol 1mw-20X; Polymer of 2-propenoic acid, cross-linked with allyl ethers of pentaerythritol

Cyclomethicone (and) Dimethiconol

Aloe Barbadenbsis Gel

Algae Extract, also known as Fucus oil; Fucus vesiculosus oil; Seaweed extract or Seaweed oil

Triethanolamine, also known as 2,2′,2″-Nitrilotriethanol; Ethanol, 2,2′,2″-nitrilotri-; TEA; Nitrilotriethanol; TEA (amino alcohol); Triethylamine, 2,2′,2″-trihydroxy-; Triethylolamine; Trihydroxytriethylamine; Tris(2-hydroxyethyl)amine; Trolamine; 2,2′,2″-Nitrilotris(ethanol); Ethanol, 2,2′,2″-nitrilotris-

Disodium EDTA, also known as Disodium N,N′-1,2-ethanediylbis(N-(carboxymethyl)glycine); Disodium edetate; Disodium ethylenediaminetetraacetate; Edetate disodium; Glycine, N,N′-1,2-ethanediylbis(N-(carboxymethyl)-, disodium salt; Edetate sodium [USAN]; Acetic acid, (ethylenedinitrilo)tetra-, disodium salt; Ethylene diamine tetraacetic acid, disodium salt

Mica, also known as Muscovite mica; Mica-group minerals; Silicate, mica

Propylene Glycol (and) Methyparaben (and) Propylparaben (and) Diazolidinyl Urea

Fragrance

c. PearLife Fantastic O's Cleanser

Water

PEG-8

Glycerin, also known as 1,2,3-Propanetriol, Glycyl alcohol; Propanetriol or Trihydroxypropane

Sodium Laureth Sulfate, also known as Sodium dodecylpoly(oxyethylene)sulfate; Sodium lauryl sulfate ethoxylate; Sodium polyoxyethylene lauryl ether sulfate; Sodium laureth-8 sulfate; Laureth-8 carboxylic acid, sodium salt; PEG-# lauryl ether sulfate, sodium salt where # is 5,7,8 or 12; Polyethylene glycol (#) lauryl ether sulfate, sodium salt where # is 5,7,12,400 or 600; Sodium laureth-# sulfate where # is 5,7 or 12; or Sodium lauryl ether sulfate

Potassium Cocoate, also known as Fatty acids, coconut oil, potassium salts; Soap; Fatty acids, coco, potassium salts; Cocoa fatty acids, potassium salts; or Potassium coconut oil soap

Sodium Lauryl Sulfate, also known as Sulfuric acid monododecyl ester sodium salt; Sodium dodecyl sulfate; Dodecyl sulfate, sodium salt; Sodium lauryl sulfate ether; Sodium n-dodecyl sulfate; or Sodium Lauryl Sulfate

Cetyl Alcohol, also known as Alcohol C-16; Cetostearyl alcohol; 1-Hexadecanol; 1-Hexadecyl alcohol; or Palmityl alcohol

Glyceryl Stearate, also known as Stearin, 1-mono-; (1)-2,3-Dihydroxypropyl stearate; Glycerol stearate, pure; Octadecanoic acid, 2,3-dihydroxypropyl ester; Stearin, or 1-mono-(8CI)

Propylene Glycol Dicaprylate/Dicaprate, also known as Decanoic acid, 1-methyl-1,2-ethanediyl ester mixed with 1-methyl-1,2-ethanediyl dioctanoate; or Carbomer 941; Carbomer

Allantoin, also known as (2,5-Dioxo-4-imidazolidinyl)urea; 5-Ureidohydantoin; Glyoxyldiureide; Hydantoin, 5-ureido-; Urea, (2,5-dioxo-4-imidazolidinyl)-

Hydroxyethyl cellulose, also known as Hydroxyethylcellulose; 2-Hydroxyethyl cellulose; 2-Hydroxyethyl cellulose ether; Cellulose, 2-hydroxyethyl ether; Cellulose hydroxyethylate; Cellulose, ethylene oxide-grafted; H. E. Cellulose; Hetastarch

Panthenol, also known as Butanamide, 2,4-dihydroxy-N-(3-hydroxypropyl)-3,3-dimethyl-, (R)-; Butyramide, 2,4-dihydroxy-N-(3-hydroxypropyl)-3,3-dimethyl-, D-(+)-; D-Panthenol; Dexpanthenol; Propanolamine, N-pantoyl-; d-Pantothenyl alcohol; Butanamide, 2,4-dihydroxy-N-(3-hydroxypropyl)-3,3-dimethyl-, (2R)-

Tocopheryl Acetate, also known as 2H-1-Benzopyran-6-ol, 3,4-dihydro-2,5,7,8-tetramethyl-2-(4,8,12-trimethyltridecyl)-, acetate; 3,4-Dihydro-2,5,7,8-tetramethyl-2-(4,8,12-trimethyltridecyl)-2H-b-enzopyran-6-ol, acetate; DL-alpha tocopheryl acetate; or Vitamin E acetate

Natural Pearl Powder

Methylparaben, also known as 4-Hydroxybenzoic acid, methyl ester; Benzoic acid, 4-hydroxy-, methyl ester; Benzoic acid, p-hydroxy-, methyl ester; Methyl 4-hydroxybenzoate; Methyl p-hydroxybenzoate; or Methyl parahydroxybenzoate

Propylparaben, also known as 4-Hydroxybenzoic acid, propyl ester; Benzoic acid, 4-hydroxy-, propyl ester; Benzoic acid, p-hydroxy-, propyl ester; Propyl 4-hydroxybenzoate; Propyl parahydroxybenzoate; Propyl p-hydroxybenzoate; or Propyl paraben

Fragrance

d. PearLife Fantastic O's Toner

Water

Polysorbate 20

Natural Pearl Powder

Glycerin, also known as 1,2,3-Propanetriol, Glycyl alcohol; Propanetriol or Trihydroxypropane

Hydroxyethyl cellulose, also known as Hydroxyethylcellulose; 2-Hydroxyethyl cellulose; 2-Hydroxyethyl cellulose ether; Cellulose, 2-hydroxyethyl ether; Cellulose hydroxyethylate; Cellulose, ethylene oxide-grafted; H. E. Cellulose; Hetastarch

Panthenol, also known as Butanamide, 2,4-dihydroxy-N-(3-hydroxypropyl)-3,3-dimethyl-, (R)-; Butyramide, 2,4-dihydroxy-N-(3-hydroxypropyl)-3,3-dimethyl-, D-(+)-; D-Panthenol; Dexpanthenol; Propanolamine, N-pantoyl-; d-Pantothenyl alcohol; Butanamide, 2,4-dihydroxy-N-(3-hydroxypropyl)-3,3-dimethyl-, (2R)-

Aloe barbadensis Leaf Juice

Hamamelis Virginiana Extract, also known as Witch hazel distillates; Extract of hamamelis; Extract of witch hazel; Hamamelis extract; or Winterbloom; Oils

Salvia Officinalis (Sage) Leaf Extract, also known as Extract of sage; Sage extract; Sage, Salvia officinalis, ext.; Salvia extract; Salvia triloba extract

Algae Extract, also known as Fucus oil; Fucus vesiculosus oil; Seaweed extract or Seaweed oil

Citric Acid, also known as 1,2,3-Propanetricarboxylic acid, 2-hydroxy-; 2-Hydroxy-1,2,3-propanetricarboxylic acid; 2-Hydroxypropanetricarboxylic acid; 2-Hydroxytricarballylic acid; 3-Carboxy-3-hydroxypentane-1,5-dioic acid

Methylparaben, also known as 4-Hydroxybenzoic acid, methyl ester; Benzoic acid, 4-hydroxy-, methyl ester; Benzoic acid, p-hydroxy-, methyl ester; Methyl 4-hydroxybenzoate; Methyl p-hydroxybenzoate; or Methyl parahydroxybenzoate

DMDM Hydantoin, also known as 2,3-Imidazolidinedione, 1,3-bis(hydroxymethyl)-5,5-dimethyl-; 1,3-Dimethylol-5,5-dimethyl hydantoin; 2,4-Imidazolidinedione, 1,3-bis(hydroxymethyl)-5,5-dimethyl-; Hydantoin, 1,3-bis(hydroxymethyl)-5,5-dimethyl-

Fragrance

e. PearLife Fantastic O's Eye Cream

Water

C12-15 Alkyl Benzoate

Ethylhexyl Palmitate

Glyceryl Stearate (and) PEG-100 Stearate Cetyl Alcohol

Dimethicone, also known as Dimethylpolysiloxane (1); Dimethyl polysiloxane; Dimethyl polysiloxane, bis(trimethylsilyl)-terminated; Polydimethylsiloxane (2); Dimethicone 350 [USAN]

Cyelomethicone

Stearic Acid, also known as 1-Heptadecanecarboxylic acid; Octadecanoic acid; or n-

Glycerin, also known as 1,2,3-Propanetriol, Glycyl alcohol; Propanetriol or Trihydroxypropane

Emulsifying Wax

Natural Pearl Powder

Hydroxyethyl cellulose, also known as Hydroxyethylcellulose; 2-Hydroxyethyl cellulose; 2-Hydroxyethyl cellulose ether; Cellulose, 2-hydroxyethyl ether; Cellulose hydroxyethylate; Cellulose, ethylene oxide-grafted; H. E. Cellulose; Hetastarch

Triethanolamine, also known as 2,2′,2″-Nitrilotriethanol; Ethanol, 2,2′,2″-nitrilotri-; TEA; Nitrilotriethanol; TEA (amino alcohol); Triethylamine, 2,2′,2″-trihydroxy-; Triethylolamine; Trihydroxytriethylamine; Tris(2-hydroxyethyl)amine; Trolamine; 2,2′,2″-Nitrilotris(ethanol); Ethanol, 2,2′,2″-nitrilotris-

DMDM Hydantoin, also known as 2,3-Imidazolidinedione, 1,3-bis(hydroxymethyl)-5,5-dimethyl-; 1,3-Dimethylol-5,5-dimethyl hydantoin; 2,4-Imidazolidinedione, 1,3-bis(hydroxymethyl)-5,5-dimethyl-; Hydantoin, 1,3-bis(hydroxymethyl)-5,5-dimethyl-

Carbomer, also known as Carbomer; 2-Propenoic acid, homopolymer; Carbomer 934 [USAN]; Carbomer 940 [USAN]; Polyacrylic acid; Acrylic polymer(s); Carbomer 941 [USAN]; Acrylic acid, polymers; Acrysol 1mw-20X; Polymer of 2-propenoic acid, cross-linked with allyl ethers of pentaerythritol

Allantoin, also known as (2,5-Dioxo-4-imidazolidinyl)urea; 5-Ureidohydantoin; Glyoxyldiureide; Hydantoin, 5-ureido-; Urea, (2,5-dioxo-4-imidazolidinyl)-

Panthenol, also known as Butanamide, 2,4-dihydroxy-N-(3-hydroxypropyl)-3,3-dimethyl-, (R)-; Butyramide, 2,4-dihydroxy-N-(3-hydroxypropyl)-3,3-dimethyl-, D-(+)-; D-Panthenol; Dexpanthenol; Propanolamine, N-pantoyl-; d-Pantothenyl alcohol; Butanamide, 2,4-dihydroxy-N-(3-hydroxypropyl)-3,3-dimethyl-, (2R)-

Tocopheryl Acetate, also known as 2H-1-Benzopyran-6-ol, 3,4-dihydro-2,5,7,8-tetramethyl-2-(4,8,12-trimethyltridecyl)-, acetate; 3,4-Dihydro-2,5,7,8-tetramethyl-2-(4,8,12-trimethyltridecyl)-2H-b-enzopyran-6-ol, acetate; DL-alpha tocopheryl acetate; or Vitamin E acetate

Salvia Officinalis (Sage) Leaf Extract, also known as Extract of sage; Sage extract; Sage, Salvia officinalis, ext.; Salvia extract; Salvia triloba extract

Anthemis Nobilis (Chamomile) Flower Extract, also known as Chamomile extract; Anthemis nobilis extract; Extract of chamomile; English chamomile extract; Roman chamomile extract

Algae Extract, also known as Fucus oil; Fucus vesiculosus oil; Seaweed extract or Seaweed oil

Retinyl Palmitate

Methylparaben, also known as 4-Hydroxybenzoic acid, methyl ester; Benzoic acid, 4-hydroxy-, methyl ester; Benzoic acid, p-hydroxy-, methyl ester; Methyl 4-hydroxybenzoate; Methyl p-hydroxybenzoate; or Methyl parahydroxybenzoate

Propylparaben

f. PearLife Fantastic O's Collagen Serum

Water

Glycerin, also known as 1,2,3-Propanetriol, Glycyl alcohol; Propanetriol or Trihydroxypropane

Soluble Collagen

Natural Pearl Powder

Hydroxyethyl cellulose, also known as Hydroxyethylcellulose; 2-Hydroxyethyl cellulose; 2-Hydroxyethyl cellulose ether; Cellulose, 2-hydroxyethyl ether; Cellulose hydroxyethylate; Cellulose, ethylene oxide-grafted; H. E. Cellulose; Hetastarch

Polysorbate 20, also known as Polysorbate 21 or 80; Liposorb; PEG sorbitan laurate; Tween 20; PEG-# sorbitan laurate (where #=10,40,44,75, or 80); Polyethylene glycol (#) sorbitan laurate (where #=4,10,40,44,75,80,500,2000, or 4000)

DMDM Hydantoin, also known as 2,3-Imidazolidinedione, 1,3-bis(hydroxymethyl)-5,5-dimethyl-; 1,3-Dimethylol-5,5-dimethyl hydantoin; 2,4-Imidazolidinedione, 1,3-bis(hydroxymethyl)-5,5-dimethyl-; Hydantoin, 1,3-bis(hydroxymethyl)-5,5-dimethyl-

Hydrolyzed

Elastin

Panthenol, also known as Butanamide, 2,4-dihydroxy-N-(3-hydroxypropyl)-3,3-dimethyl-, (R)-; Butyramide, 2,4-dihydroxy-N-(3-hydroxypropyl)-3,3-dimethyl-, D-(+)-; D-Panthenol; Dexpanthenol; Propanolamine, N-pantoyl-; d-Pantothenyl alcohol; Butanamide, 2,4-dihydroxy-N-(3-hydroxypropyl)-3,3-dimethyl-, (2R)-

Aloe Barbadensis Leaf Juice

Algae Extract, also known as Fucus oil; Fucus vesiculosus oil; Seaweed extract or Seaweed oil

Citric Acid, also known as 1,2,3-Propanetricarboxylic acid, 2-hydroxy-; 2-Hydroxy-1,2,3-propanetricarboxylic acid; 2-Hydroxypropanetricarboxylic acid; 2-Hydroxytricarballylic acid; 3-Carboxy-3-hydroxypentane-1,5-dioic acid

Methylparaben, also known as 4-Hydroxybenzoic acid, methyl ester; Benzoic acid, 4-hydroxy-, methyl ester; Benzoic acid, p-hydroxy-, methyl ester; Methyl 4-hydroxybenzoate; Methyl p-hydroxybenzoate; or Methyl parahydroxybenzoate

Fragrance

g. PearLife Fantastic O's Vitamin C Powder

Natural Pearl Powder

Purified Water

Propylene Glycol, also known as 1,2-Dihydroxypropane; 1,2-Propylene glycol; 2,3-Propanediol; Isopropylene glycol; 1,2-Propanediol;or Trimethyl glycol Glycerin, also known as 1,2,3-Propanetriol, Glycyl alcohol; Propanetriol or Trihydroxypropane

Silica Silylate

Polymethylsilsesquioxane

Methylparaben, also known as 4-Hydroxybenzoic acid, methyl ester; Benzoic acid, 4-hydroxy-, methyl ester; Benzoic acid, p-hydroxy-, methyl ester; Methyl 4-hydroxybenzoate; Methyl p-hydroxybenzoate; or Methyl parahydroxybenzoate

Imidazolidinyl Urea, also known as Imidurea; Urea, N,N″-methylenebis(N′-(3-(hydroxymethyl)-2,5-dioxo-4-imidazolidinyl)-; N,N″-Methylenebis(N′-(1-(hydroxymethyl)-2,5-dioxo-4-imidazolidinyl)urea)

h. PearLife Fantastic O's Arbutin Powder

Water

Glycerin, also known as 1,2,3-Propanetriol, Glycyl alcohol; Propanetriol or Trihydroxypropane

Silica Silylate

Vunyl Dimethicone/Methicone

Silesquiozane Crosspolymer

Arbutin

Methylparaben, also known as 4-Hydroxybenzoic acid, methyl ester; Benzoic acid, 4-hydroxy-, methyl ester; Benzoic acid, p-hydroxy-, methyl ester; Methyl 4-hydroxybenzoate; Methyl p-hydroxybenzoate; or Methyl parahydroxybenzoate

Imidazolidinyl Urea, also known as Imidurea; Urea, N,N″-methylenebis(N′-(3-(hydroxymethyl)-2,5-dioxo-4-imidazolidinyl)-; N,N″-Methylenebis(N′-(1-(hydroxymethyl)-2,5-dioxo-4-imidazolidinyl)urea)

Hamamelis Virginiana Extract, also known as Witch hazel distillates; Extract of hamamelis; Extract of witch hazel; Hamamelis extract; or Winterbloom; Oils

SD Alcohol 39-C

Butylene Glycol, also known as 1,3-Butanediol; or 1,3-Butylene glycol

Dipotassium Glycyrrhizate

Allantoin, also known as (2,5-Dioxo-4-imidazolidinyl)urea; 5-Ureidohydantoin; Glyoxyldiureide; Hydantoin, 5-ureido-; Urea, (2,5-dioxo-4-imidazolidinyl)-

Tocopheryl Acetate, also known as 2H-1-Benzopyran-6-ol, 3,4-dihydro-2,5,7,8-tetramethyl-2-(4,8,12-trimethyltridecyl)-, acetate; 3,4-Dihydro-2,5,7,8-tetramethyl-2-(4,8,12-trimethyltridecyl)-2H-b-enzopyran-6-ol, acetate; DL-alpha tocopheryl acetate; or Vitamin E acetate

1. A topical application comprising: cultured pearl powder of particle size ranging between 0.4 μm and 1 μm ground by an ultrasonic gas jet mill, and purified water suspending the cultured pearl powder.

2. The topical application of claim 1 further comprising: glycerin, C12-15 Alkyl benzoate, ethylhexyl palmitate, ethythexyl methoxycinnamate, glyceryl stearate, PEG-100 stearate, cetyl alcohol, and stearic acid, wherein the topical application forms a cream consistency for day use.

3. The topical application of claim 1 further comprising: Butylenes Glycol, Panthenol, Ethylhexyl Palmitate, C12-15 Alkyl Benzoate, Glyceryl Stearate, Peg-100 Stearate, Cetyl Alcohol, Stearic Acid, Emusifying Wax, Dimethicone, Tocopheryl Acetate, Retinyl Palmitate, Carbomer, Cyclomethicone, Dimethiconol, Aloe Barbadenbsis Gel, Algae Extract, Triethanolamine, Disodium EDTA, Mica, Propylene Glycol, Methyparaben, Propylparaben, Diazolidinyl Urea, Fragrance, wherein the topical application forms a cream consistency for use as a night cream.

4. The topical application of claim 1 further comprising: PEG-8, Glycerin, Sodium Laureth Sulfate, Potassium Cocoate, Sodium Lauryl Sulfate, Cetyl Alcohol, Glyceryl Stearate, Propylene Glycol Dicaprylate/Dicaprate, Allantoin, Hydroxyethylcellulose, Panthenol, Tocopheryl acetate, Methylparaben, Propylparaben, Fragrance, wherein the topical application forms a consistency for use as a cleanser.

5. The topical application of claim 1 further comprising: Polysorbate 20, Glycerin, Hydroxyethylcellulose, Panthenol, Aloe barbadensis Leaf Juice, Hamamelis Virginiana (Witch Hazel) Extract, Salvia Officinalis (Sage) Leaf Extract, Algae Extract, Citric Acid, Methylparaben, DMDM Hydantoin, Fragrance, wherein the topical application forms a consistency for use as a toner.

6. The topical application of claim 1 further comprising: C12-15 Alkyl Benzoate, Ethylhexyl Palmitate, Glyceryl Stearate, PEG-100 Stearate, Cetyl Alcohol, Dimethicone, Cyclomethicone, Stearic Acid, Glycerin, Emulsifying Wax, Hydrozyethylcellulose, Triethanolamine, DMDM Hydantoin, Carbomer, Allantoin, Panthenol, Tocopheryl Acetate, Salvia Officinalis (Sage) Leaf Extract, Anthemis Nobilis (Chamomile) Flower Extract, Algae Extract, Retinyl Palmitate, Methylparaben, Propylparaben, wherein the topical application forms a consistency for use as an eye cream.

7. The topical application of claim 1 further comprising: Glycerin, Soluble Collagen, Hydroxyethlcellulose, Polysorbate 20, DMDM Hydantoin, Elastin, Panthenol, Aloe Barbadensis Leaf Juice, Algae Extract, Citric Acid, Methyparaben, fragrance, wherein the topical application forms a consistency for use as a collagen serum.

8. The topical application of claim 1 further comprising: Propylene Glycol, Glycerin, Silica Silyate, Polymethylsilsesquioxane, Methylparaben, Imidazolidinyl Urea, wherein the topical application forms a consistency for use as a Vitamin cream.

9. The topical application of claim 1 further comprising: Glycerin, Silica Silylate, Vunyl Dimethicone/Methicone, Silesquiozane Crosspolymer, Arbutin, Methylparaben, Imidazolidinyl Urea, Hamamelis Virginiana Extract, SD Alcohol 39-C, Butylene Glycol, Dipotassium Glycyrrhizate, Allantoin, Tocopherol Acetate, wherein the topical application forms a consistency for use as a skin cream.

10. A method for making a radiation blocking topical application comprising the steps of: pulverizing pearls using supersonic gas jet pulverization machines creating 0.4 μm to 1 μm diameter pearl powder; and mixing pearl powder with water based lotion ingredients forming a radiation blocking composition.

11. The method of claim 10, further comprising the step of selecting fine fresh water pearls before the pulverizing step, wherein said pearl powder contains 82%-92% of calcium carbonate, 1% of trace elements, and 4%-14% scleroproteins.

12. The product produced by the process of claim 10.

13. The method for making a topical application of claim 10 wherein lotion ingredients further comprise: glycerin, C12-15 Alkyl benzoate, ethylhexyl palmitate, ethythexyl methoxycinnamate, glyceryl stearate, PEG-100 stearate, cetyl alcohol, and stearic acid, wherein the step of mixing forms a cream consistency for day use.

14. The method for making a topical application of claim 10 wherein lotion ingredients further comprise: Butylenes Glycol, Panthenol, Ethylhexyl Palmitate, C12-15 Alkyl Benzoate, Glyceryl Stearate, Peg-100 Stearate, Cetyl Alcohol, Stearic Acid, Emusifying Wax, Dimethicone, Tocopheryl Acetate, Retinyl Palmitate, Carbomer, Cyclomethicone, Dimethiconol, Aloe Barbadenbsis Gel, Algae Extract, Triethanolamine, Disodium EDTA, Mica, Propylene Glycol, Methyparaben, Propylparaben, Diazolidinyl Urea, Fragrance, wherein the step of mixing forms a cream consistency for use as a night cream.

15. The method for making a topical application of claim 10 wherein lotion ingredients further comprise: PEG-8, Glycerin, Sodium Laureth Sulfate, Potassium Cocoate, Sodium Lauryl Sulfate, Cetyl Alcohol, Glyceryl Stearate, Propylene Glycol Dicaprylate/Dicaprate, Allantoin, Hydroxyethylcellulose, Panthenol, Tocopheryl acetate, Methylparaben, Propylparaben, Fragrance, wherein the step of mixing forms a consistency for use as a cleanser.

16. The method for making a topical application of claim 10 wherein lotion ingredients further comprise: Polysorbate 20, Glycerin, Hydroxyethylcellulose, Panthenol, Aloe barbadensis Leaf Juice, Hamamelis Virginiana (Witch Hazel) Extract, Salvia Officinalis (Sage) Leaf Extract, Algae Extract, Citric Acid, Methylparaben, DMDM Hydantoin, Fragrance, wherein the step of mixing forms a consistency for use as a toner.

17. The method for making a topical application of claim 10 wherein lotion ingredients further comprise: C12-15 Alkyl Benzoate, Ethylhexyl Palmitate, Glyceryl Stearate, PEG-100 Stearate, Cetyl Alcohol, Dimethicone, Cyclomethicone, Stearic Acid, Glycerin, Emulsifying Wax, Hydrozyethylcellulose, Triethanolamine, DMDM Hydantoin, Carbomer, Allantoin, Panthenol, Tocopheryl Acetate, Salvia Officinalis (Sage) Leaf Extract, Anthemis Nobilis (Chamomile) Flower Extract, Algae Extract, Retinyl Palmitate, Methylparaben, Propylparaben, wherein the step of mixing forms a consistency for use as an eye cream.

18. The method for making a topical application of claim 10 wherein lotion ingredients further comprise: Glycerin, Soluble Collagen, Hydroxyethlcellulose, Polysorbate 20, DMDM Hydantoin, Elastin, Panthenol, Aloe Barbadensis Leaf Juice, Algae Extract, Citric Acid, Methyparaben, fragrance, wherein the step of mixing forms a consistency for use as a collagen serum.

19. The method for making a topical application of claim 10 wherein lotion ingredients further comprise: Propylene Glycol, Glycerin, Silica Silyate, Polymethylsilsesquioxane, Methylparaben, Imidazolidinyl Urea, wherein the step of mixing forms a consistency for use as a Vitamin cream.

20. The method for making a topical application of claim 10 wherein lotion ingredients further comprise: Glycerin, Silica Silylate, Vunyl Dimethicone/Methicone, Silesquiozane Crosspolymer, Arbutin, Methylparaben, Imidazolidinyl Urea, Hamamelis Virginiana Extract, SD Alcohol 39-C, Butylene Glycol, Dipotassium Glycyrrhizate, Allantoin, Tocopherol Acetate, wherein the step of mixing forms a consistency for use as a skin cream.
